(19) United States
(12) Patent Application Publication
(10) Pub. No.: US 2010/0150851 A1
(43) Pub. Date: Jun. 17, 2010

(54) ANIONIC-CATIONIC COMPLEXES AS CONDITIONING AGENTS

(75) Inventors: Albert A. Zofchak, Holmdel, NJ (US); John Carson, Union City, NJ (US); Paul Slavashevich, Bronx, NY (US)

Correspondence Address: COLEMAN SUDOL SAPONE, P.C., 714 COLORADO AVENUE, BRIDGE PORT, CT, 06605-1601, US

(73) Assignee: ALZO INTERNATIONAL, INC., Sayreville, NJ (US)

(21) Appl. No.: 12/450,865

(22) PCT Filed: Apr. 11, 2008
(86) PCT No.: PCT/US2008/004669

§ 371 Date: Mar. 1, 2010

Related U.S. Application Data
(60) Provisional application No. 60/925,967, filed on Apr. 24, 2007.

(51) Int. Cl. A61K 8/36; C07C 57/13; A61K 8/87; A61Q 9/02; A61Q 5/02; A61Q 5/12; A61Q 1/06; A61Q 17/04; A61Q 15/00

(52) U.S. Cl. 424/59; 562/595; 424/70.11; 424/73; 424/70.1; 424/64; 424/65; 514/554

(57) ABSTRACT

Novel compositions comprising anionic-cationic complexes as conditioning agents for use in products that improve the appearance and feel of skin and hair are well known. This invention describes the use of additional cationic surfactant in combination with an anionic-cationic complex made from a fatty alkyl dialkylamine and a “dicarboxylic acid”, preferably a dimer acid. This combination provides exceptional conditioning for hair, improving both wet and dry combing. It also makes very stable oil-in-water emulsions, and adds a slippery feel and lubricity to shaving and skin cleaning products.

### RELATED APPLICATIONS

This patent application claims the benefit of priority of provisional application number U.S. 60/925,967, filed Apr. 24, 2007, entitled “Anion-Cationic Complexes as Conditioning Agents”, which is incorporated by reference in its entirety herein.

### FIELD OF THE INVENTION

The present invention relates to compositions comprising a dimer acid amine complex or salt and a quaternary ammonium condition and their use as conditioning agents. These compositions may be used as conditioners in shampoos and conditioning agents or to produce and stabilize water-in-oil emulsions which are included in personal care products. The compositions according to the present invention exhibit exceptional conditioning for hair, improving both wet and dry combing and static control features. The present compositions also can be used to produce stable water-in-oil emulsions, which add a slippery feel and lubricity to numerous personal care products such as shaving creams/gels and skin cleaning products, among numerous others.

### BACKGROUND OF THE INVENTION

Many personal care products have been formulated using the anionic-cationic complexes sold under the Necon brand name by Alzo International, Inc. These products have been used for many years as hair conditioning agents, thickeners, lubricants for the skin, emulsifiers and emulsion stabilizers. The Necons are the reaction products of various fatty acids and various fatty amines. The different combinations of fatty acids and fatty amines allow the creation of a wide range of products each having different properties. This allows the formulation of novel personal care and cosmetic products through the use of a Necon product that has a specific property that is desirable in that particular formulation. Thus, the formulas, and the Necons, can be optimized to provide specific properties. For example, the formulation of a shaving cream that has exceptional slip due to the lubricating properties of Necon LO (INCI name—Dimethyl Lauramine Oleate). Many of these applications are detailed in U.S. Pat. No. 4,548,810 that discusses the formation of the fatty acid, fatty amine reaction products. In addition, U.S. Pat. No. 6,723,310 discusses the use of several different combinations of fatty acids and fatty amines that can be used in shampoo formulations to repair split ends in damaged hair. In that patent, the combination of dimer dilinoleic acid with lauryldimethylamine (Necon DLD) was found to be a preferred material for repairing split ends from a shampoo formula.

The current studies began with the intent of evaluating the split end mending properties of Necon DLD when used in hair conditioner formulas. It was felt that there might be a greater split end repair effect if the Necon DLD conditioner were to be applied directly to clean, but still wet hair, after it is shampooed. In addition, as a conditioner, the product could be evenly distributed throughout the hair or concentrated in specific areas that are highly damaged. It was also felt that, because there are no anionic shampoo surfactants present to “wash” the conditioner from the hair or alter its effects, enhanced split end repair should occur. What was surprisingly found was a hair conditioner formula that noticeably improved the wet combing and detangling of hair. In addition, the dry hair combing and static control attributes were improved.

Hair conditioners are traditionally made with fatty, cationic surfactants such as stearyl dimethyl benzyl ammonium chloride, distearyl dimethyl ammonium chloride, behenyl trimethyl ammonium chloride, behenyl trimethyl ammonium methosulfate, behenamidopropyl dimethyl ethyl ammonium ethosulfate and the like. The use level of the “active” quaternary surfactant is from about 0.5% to about 5%. Cationic hair conditioners made with behenyl trimethyl ammonium chloride, behenyl trimethyl ammonium methosulfate and behenamidopropyl dimethyl ethyl ammonium ethosulfate are especially noted for their improved dry hair combing, manageability and antistatic effects. These effects are very desirable.

The hair conditioner formulas often also contain fatty alcohols (such as cetyl and stearyl alcohols) to increase viscosity, improve the emulsifying ability, add tactile feel and “substance” to the conditioner, improve the manageability of the hair and to make the conditioner opaque. The conditioners will also often contain oils (such as: mineral oil, silicone oil and vegetable oils) to add lubricity and shine to the hair. Finally, cationic, hydrophilic polymers (such as INCI names: Polyquaternium 10 and Guar Hydroxypropyltrimonium Chloride) are often added to reduce wet combing friction and improve hair detangling thereby making wet hair easier to comb.

Conventional hair conditioner formulas do not contain anionic materials because they form complexes with the cationic “conditioning” surfactants thereby inactivating them or causing them to precipitate. It has been surprisingly found that anionic materials (such as dimer dilinoleic acid) can be used in hair conditioner formulas by using the amine reaction product (for example Necon DLD) and that the resulting formulas are stable and highly effective hair conditioners.

### OBJECTS OF THE INVENTION

It is an object of this invention to produce a hair conditioner formulation having improved hair detangling when compared to commonly used hair conditioner quaternary surfactants such as stearalkonium chloride. This is accomplished through the use of Necon DLD or a related compound in combination with a quaternary amine surfactant such as behenyl amidopropyl dimethyl ethyl ammonium ethosulfate and/or stearyl amidopropyl dimethyl ethyl ammonium ethosulfate.

It is an object of this invention to produce a blend of Necon DLD or a related compound, behenyl amidopropyl dimethyl ethyl ammonium ethosulfate and/or stearyl amidopropyl dimethyl ethyl ammonium ethosulfate or other quaternary amine surfactant and fatty alcohol that is suitable for use as a conditioner base when combined with water and further is capable of forming emulsions when combined with suitable oily materials and water.

It is a further object of this invention to produce a gel system that is capable of thickening water based surfactant systems and providing hair conditioning through the use of Necon DLD, behenyl amidopropyl dimethyl ethyl ammonium ethosulfate and/or stearyl amidopropyl dimethyl ethyl ammonium ethosulfate and Steareth-75 IPDI or other polyurethane and water.

It is an object of this invention to use the combination of Necon DLD or a related compound, behenyl amidopropyl dimethyl ethyl ammonium ethosulfate and/or stearyl amidopropyl dimethyl ethyl ammonium ethosulfate or a related quaternary amine surfactant and Steareth-75 IPDI, Steareth-100 IPDI or other diurethane and water as a thickener and conditioning agent in surfactant based personal cleaning products such as shampoos and body washes.

It is a further object of this invention to produce a cationic emulsifying system that will quickly and easily emulsify oils into water producing stable emulsions that produce a very lubricious feel on skin for use in skin care products.

It is a further object of this invention to produce shaving products having a highly lubricious lather or emulsion that greatly reduces the friction and discomfort of shaving.

### BRIEF DESCRIPTION OF THE FIGURES

FIG. 1 shows the chemical structure of a number of components used in the present invention.

### SUMMARY OF THE INVENTION

The present invention is directed to a composition comprising an effective amount of dicarboxylic acid, preferably a dimer acid, complexed with a tertiary monofattytrialkylamine (dimer acid amine complex or salt) in a molar ratio of about 1:2 wherein said monofattytrialkylamine has the formula I:

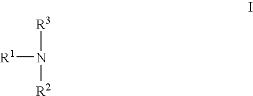

Where R¹ is a C₈ to C₂₄ alkyl group (preferably a C₁₂ to C₁₈ alkyl group);
R² and R³ are each independently a C₁-C₃ alkyl group to form a dimer acid amine salt compound according to formula II:

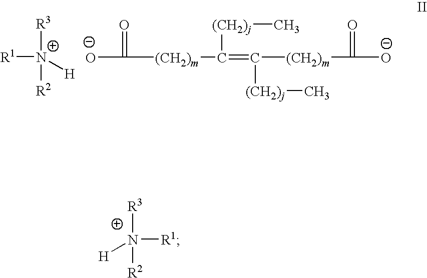

Where R¹, R² and R³ are the same as above; and each m is from 4 to 12, preferably 6-10, most preferably 7, and each j is from 4 to 12, most preferably 8; and
A quaternary ammonium conditioner having at least one C₈-C₂₀ fatty alkyl group;
Wherein the ratio of said dimer acid amine salt compound (preferably, the compound of formula II) to said quaternary ammonium conditioner in said composition ranges from about 10:1 to about 1:10. In additional aspects of the present invention, the composition according to the present invention includes water and optional and preferable effective amount of fatty (C₁₄-C₂₂ alkyl, preferably a C₁₈ alkyl or stearyl capped) end-capped polyethyleneoxide polyurethane thickener such as disteareth 75 IPDI, disteareth 100 IPDI, and related thickeners.

In preferred embodiments, the dimer acid is dimer dilinoleic acid such as that in the compound of formula II, m is 7 and j is 8.

In preferred aspects of the invention, the quaternary amine conditioner has the structure:

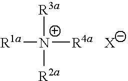

Where R^{1a} is a —(CH₂)ₙ—N_{y}—Z group;
n is 0, 1, 2, 3, 4, 5, 6, 7 or 8;
y is 0 or 1, with the proviso that y is 0 when n is 0; and
Z is a C₈-C₂₄ acyl group or alkyl group, preferably an acyl group;
R^{2a} and R^{3a} and each independently a C₁-C₃ alkyl group;
R^{4a} is a group formed by reacting R^{1a}R^{2a}R^{3a}N with a quaternizing agent selected from the group consisting of dimethyl sulfate, diethyl sulfate, methyl chloride, methyl bromide, benzyl chloride, ethyl benzyl chloride, methyl benzyl chloride, dichloroethyl ether, epichlorohydrin, ethylene chlorohydrin, methyl chloride and allyl chloride to form an N—R^{4a} group with the amine and the resulting positively charged quaternary amine group is complexed with an anionic group or counterion, which is represented as X⁻. The counterion may be any group which is anionic and is compatible with the chemistry of the present invention and preferably is an anionic chloride, bromide, iodide, fluoride, carboxylate (from, for example the use of chloroacetic acid or sodium monochloroacetate as the quaternizing agent to provide an acetate which can provide both a quaternium group as well as the counterion), sulfate (mono- or di-anion, preferably alkyl substituted mono-anion such as methyl or ethyl sulfate, more preferably ethyl sulfate or ethosulfate group [CH₃CH₂OSO₃⁻] or phosphate (mono-, di- and tri-anion, preferably tri-anion), among numerous others, with anionic chloride and sulfate (alkyl substituted mono-anion—especially ethosulfate) being the preferred counterion X. In particularly preferred aspects of the invention, the quaternary amine surfactant is isostearyl dimethylethylammonium ethosulfate (Foamquat IAES) and/or behenyl aminopropyldimethylethylammonium ethosulfate (Foamquat BAS).

In certain preferred embodiments of the present compositions, the dimer acid amine salt and the quaternary amine surfactant is combined with an effective amount (about 0.01% to about 5%, about 0.1% to about 2.5% about 0.25% to about 2%, about 0.5% to about 1.5%) of a fatty polyethyleneoxide polyurethane thickener, preferably steareth 75 IPDI (stearyl end-capped to about 37 polyethylene oxide groups on each side of an isophorone diurethane) or steareth 100 IPDI (stearyl end-capped to about 50 polyethylene oxide groups on each side of an isophorone diurethane) which is formed by reacting a stearyl end-capped polyethylene oxide group to isophorone diisocyanate.

### DETAILED DESCRIPTION OF THE INVENTION

The following definitions shall be used in describing the present invention.

The term “personal care product” is used throughout the specification to describe a cosmetic or toiletry product which is preferably used on or in contact with the hair, skin and/or nails and which include effective concentrations of one or more of the compositions according to the present invention. Personal care products include, for example, cosmetics, floating bath oils, after shaves, creams, lotions, deodorants, including stick deodorants, pre-electric shave lotions, after-shave lotions, antiperspirants, shampoos, conditioners and rinses and related products, among others, including skin care products, eye makeups, body shampoos, protective skin formulations, lipsticks, lip glosses, after-bath splashes, presun and sun products, including sunscreens. Preferred personal care products include shampoos, conditioners, rinses and related haircare products. Virtually any chemical product which comes into contact with the hair or skin and which may include effective amounts or concentrations of one or more of the compositions according to the present invention may be considered a personal care product according to the present invention. Personal care product compositions comprise a dimer acid amine salt and a quaternary amine conditioning agent in effective amounts wherein the weight ratio of dimer acid amine salt to quaternary amine conditioning agent ranges from about 10:1 to about 1:10, water and optionally, an effective amount of a C₁₄-C₂₂ fatty alkyl (preferably a C₁₋₈ alkyl or stearyl capped) capped polyethyleneoxide diurethane thickener such as disteareth 75 IPDI, disteareth100 IPDI, and related alkyl capped polyoxyalkylene diurethane thickeners. Additional components may also be included in personal care products according to the present invention for example, one or more of fragrances, emollients, solvents/diluents, opacifiers, sunscreen agents, anti-perspirants, deodorizers, antiperspirants, antimicrobial agents, dyes, pigments, foaming agents, gelling agents, solubilizing agents, humectants, stiffening agents and mixtures of these components among numerous others.

The term “diisocyanate” is used throughout the specification to describe a linear, cyclic or branch-chained hydrocarbon having two free isocyanate groups, which, when reacted with an excess of an alcohol, forms a diurethane molecule. The term “diisocyanate” also includes halogen substituted linear, cyclic or branch-chained hydrocarbons having two free isocyanate groups, but preferably the diisocyanate, if substituted, is substituted with one or more C₁-C₃ alkyl groups. Exemplary diisocyanates include, for example, isophoronediisocyanate, m-phenylene-diisocyanate, p-phenylenediisocyanate, 4,4-butyl-m-phenylene-diisocyanate, 4-methoxy-m-phenylenediisocyanate, 4-phenoxy-m-phenylenediisocyanate, 4-chloro-m-phenyldiisocyanate, toluenediisocyanate, m-xylylenediisocyanate, p-xylylenediisocyanate, 1,4-napthalenediisocyanate, cumene-1,4-diisocyanate, durene-diisocyanate, 1,5-napthylenediisocyanate, 1,8-napthylenediisocyanate, 1,5-tetrahydronapthylenediisocyanate, 2,6-napthylenediisocyanate, 1,5-tetrahydronapthylenediisocyanate; p,p-diphylenediisocyanate; 2,4-diphenylhexane-1,6-diisocyanate; methylenediisocyanate; ethylenediisocyanate; trimethylenediisocyanate, tetramethylenediisocyanate, pentamethylenediisocyanate, hexamethylenediisocyanate, nonamethylenediisocyanate, decamethylene-diisocyanate, 3-chloro-trimethylenediisocyanate and 2,3-dimethyltetramethylenediisocyanate, among numerous others. Isophorone diisocyanate is used the preferred diisocyanate used in the present invention. In certain aspects of the present invention a diisocyanate, preferably isophorone diisocyanate is reacted with a fatty capped polyethylene glycol such as steareth 75 or steareth 100 to produce steareth 75 isophorone diurethane (Steareth 75 IPDI) or steareth 100 isophorone diurethane (Steareth 100 IPDI).

The term “effective” means an amount of a component, compound or composition according to the present invention included in an amount to effect an intended result, i.e., the result intended from the inclusion of the component compound or composition.

The term “emulsion”, “oil-in-water emulsion” and “water-in-oil emulsion” are used synonymously throughout the specification to describe compositions according to the present invention. An “emulsion” according to the present invention is a cream or lotion which is generally formed by the suspension of a very finely divided liquid, in this case water, in another liquid, in this case, an oil, or alternatively, an oil, in water. In the present invention, an emulsion is formed when the water phase is compatibilized in the oil phase, such that the water phase becomes dispersed within the oil phase. While not being limited by way of theory, it is believed that in the water-in-oil emulsion compositions according to the present invention, the oil phase produces a liposome- or encapsulation-like structure or a related structure surrounding water and/or the water phase, with the combination of the dimer acid amine salt and quaternary amine conditioner serving to enhance the formation of the liposome-like structure and consequently, the emulsion composition. The term emulsion is used to distinguish the present compositions from compositions which contain at least two distinct phases, i.e., an oil phase and a water phase.

The term “oil” is used throughout the specification to describe any of various lubricious, hydrophobic and combustible substances obtained from animal, vegetable and mineral matter. Oils for use in the present invention may include petroleum-based oil derivatives such as purified petrolatum and mineral oil. Petroleum-derived oils include aliphatic or wax-based oils, aromatic or asphalt-based oils and mixed base oils and may include relatively polar and non-polar oils. “Non-polar” oils are generally oils such as petrolatium or mineral oil or its derivatives which are hydrocarbons and are more hydrophobic and lipophilic compared to synthetic oils, such as esters, which may be referred to as “polar” oils. It is understood that within the class of oils, that the use of the terms “non-polar” and “polar” are relative within this very hydrophobic and lipophilic class, and all of the oils tend to be much more hydrophobic and lipophilic than the water phase which is used in the present invention.

In addition to the above-described oils, certain essential oils derived from plants such as volatile liquids derived from flowers, stems and leaves and other parts of the plant which may include terpenoids and other natural products including triglycerides may also be considered oils for purposes of the present invention.

Petrolatum (mineral fat, petroleum jelly or mineral jelly) and mineral oil products for use in the present invention may be obtained from a variety of suppliers. These products may range widely in viscosity and other physical and chemical characteristics such as molecular weight and purity. Preferred petrolatum and mineral oil for use in the present invention are those which exhibit significant utility in cosmetic and pharmaceutical products. Cosmetic grade oils are preferred oils for use in the present invention.

Additional oils for use in the present invention may include, for example, mono-, di- and tri-glycerides which may be natural or synthetic (derived from esterification of glycerol and at least one organic acid, saturated or unsaturated, such as for example, such as acetic, propionic, butyric, caproic, palmitic, stearic, oleic, linoleic or linolenic acids, among numerous others, preferably a fatty organic acid, comprising between 8 and 26 carbon atoms). Glyceride esters for use in the present invention include vegetable oils derived chiefly from seeds or nuts and include drying oils, for example, linseed, iticica and tung, among others; semi-drying oils, for example, soybean, sunflower, safflower and cottonseed oil; non-drying oils, for example castor and coconut oil; and other oils, such as those used in soap, for example palm oil. Hydrogenated vegetable oils also may be used in the present invention. Animal oils are also contemplated for use as glyceride esters and include, for example, fats such as tallow, lard and stearin and liquid fats, such as fish oils, fish-liver oils and other animal oils, including sperm oil, among numerous others. In addition, a number of other oils may be used, including C₁₂ to C₃₀ (or higher) fatty esters (other than the glyceride esters, which are described above) or any other acceptable cosmetic emollient.

Preferred oils for use in the present invention include petrolatum, mineral oil or mixtures of petrolatum and mineral oil where the amount of petrolatum to mineral oil (on a weight/weight basis) ranges from about 1:20 to about 10:1, preferably about 1:5 to about 5:1, more preferably about 1:3 to about 1:1, depending upon the end use of the emulsion composition. The inclusion of petrolatum and/or mineral oil and/or the ratio of petrolatum to mineral oil in the present compositions will greatly influence the final viscosity of the water-in-oil compositions according to the present invention. Emulsions according to the present invention comprise water in an amount ranging from about 25% to about 90%, about 35% to about 85%, about 40% to about 80%, about 45% to about 75% by weight and an oil in an amount ranging from about 5% to about 65%, about 10% to about 50%, about 15% to about 50%, an effective amount of a combination of dimer acid amine salt and quaternary amine conditioner (the combination of dimer acid amine salt and quaternary amine conditioner generally ranging from about 0.1% to about 25% by weight of the emulsion) in a weight ratio of about 10:1 to about 1:10, preferably about 3:1 to about 1:3). In addition to the above components, additional components may be added to the emulsion including fragrances, emollients, solvents/diluents, opacifiers, sunscreen agents, anti-perspirants, deodorizers, antiperspirants, antimicrobial agents, dyes, pigments, foaming agents, gelling agents, solubilizing agents, humectants, stiffening agents and mixtures of these components, among numerous other components which may be added to personal care products (compositions).

In general, compositions according to the present invention are included in personal care products/formulations in effective amounts, i.e., amounts which produce an intended effect. The amount of conditioning agents according to the present invention included in personal care products/composition is at least about 0.01% by weight, and generally ranges from about 0.05% to about 50% by weight or more of personal care formulations according to the present invention. In preferred embodiments of emulsion-based formulations, conditioning agents according to the present invention may be included in amounts ranging from about 0.05% to about 15% by weight, about 0.1% to about 10% by weight, about 0.5% to about 7.5% by weight, about 0.75% to about 5% by weight, about 1.0% to about 5% by weight, about 1% to about 3.5% by weight. In the case of shampoos and conditioners, conditioning agents according to the present invention are included in amounts ranging from about 0.05% to about 10% by weight of the formulation.

For example, in shampoos, rinses, conditioners, hair straighteners, hair colorants and permanent wave formulations, conditioning agents according to the present invention comprise about 0.05% to about 10% by weight, about 0.25% to about 3% by weight of the final end-use composition. Other components which may be included in these formulations include standard components generally used in these formulations including, for example, a solvent or diluent such as water and/or alcohol, other surfactants, thickeners, coloring agents, dyes, emollients, oils, preservatives, additional conditioning agents and humectants, among numerous others.

In the case of creams and gels, including shave lotions and shave-conditioning compositions (for example, pre-electric shave formulations), conditioning agents according to the present invention are included in amounts ranging from about 0.025% to about 15% or more by weight, about 0.1% to about 10% by weight more preferably about 0.5% to about 3% by weight. Other components which may be included in these end-use personal care compositions include, for example, water, and at least one or more of emollients, humectants and emulsifiers and optionally, other conditioning agents, medicaments, fragrances and preservatives.

In the case of skin lotions and color cosmetics, conditioning agents according to the present invention are included in amounts ranging from about 0.25% to about 15% by weight, about 0.5 to about 10% by weight, about 0.75% to about 3% by weight. Additional components which may be employed in these compositions include, for example, water, emollients, dyes, coloring agents, humectants and emulsifiers and optionally, other conditioning agents, medicaments, fragrances and preservatives.

In the case of sunscreens and skin-protective compositions, conditioning agents according to the present invention are included in amounts ranging from about 0.025% to about 15%, about 0.5% to about 7.5% by weight, about 0.5% to about 5% of the final formulations. Additional components which may be employed in these compositions may include, for example, a UV absorbing composition such as para-amino benzoic acid (PABA) or a related UV absorber or a pigment such as TiO₂, water or oil, and optional components including, for example, one or more of an oil, water, suspending agents, other conditioning agents, humectants and emollients, among others.

In the case of bar and liquid soaps, bath oils and body washes, conditioning agents according to the present invention may be included in amounts ranging from about 0.25% to about 20% by weight or more, preferably about 0.5% to about 10% by weight. Additional components which may be included in bar and liquid soaps include water, optional solvents such as ethyl alcohol or isopropanol and surfactants, and optionally, bactericides, emollients, fragrances and colorants, among others.

Conditioning agents according to the present invention may be supplied as dry powders or in solution/dispersions, wherein the cationic silicone polymer comprises up to about 75% or more by weight (preferably, 50% by weight or less) in combination with water and/or a cosmetically acceptable solvent such as ethanol and/or isopropanol.

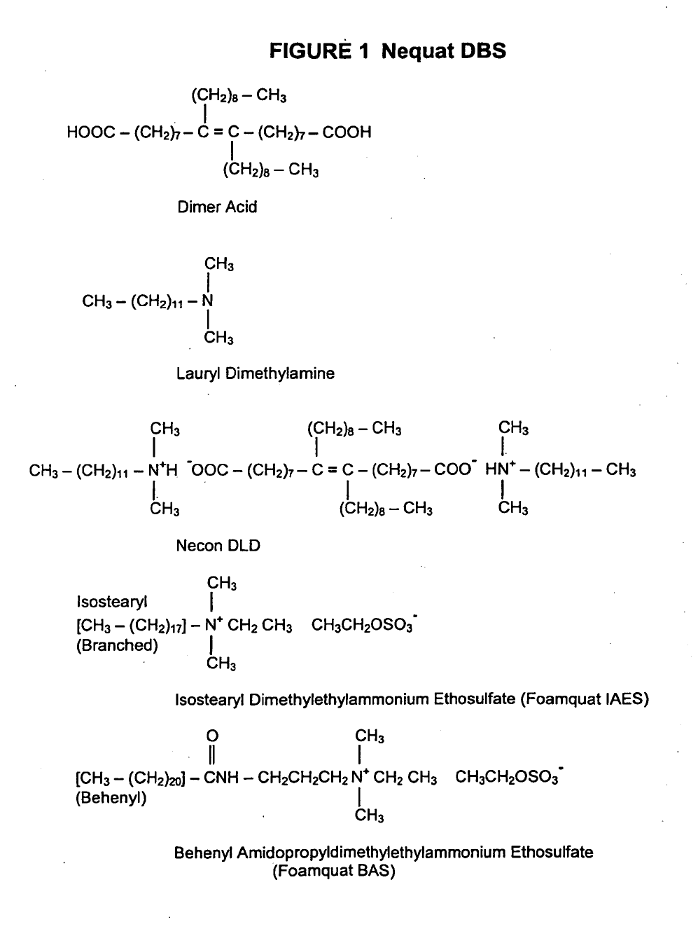

By way of example, a preferred embodiment of this invention comprises Necon DLD (see FIG. 1) combined with a cationic (quaternary amine conditioner) conditioner to enhance deposition and improve wet combing. Several cationic conditioners were tested with the preferred materials being behenamidopropyl dimethyl ethyl ammonium ethosulfate and stearamidopropyl dimethyl ethyl ammonium ethosulfate as well as combinations of these two cationics. The ingredients were dispersed and melted in stearyl alcohol. This mixture was found to be a superior hair conditioning combination providing excellent wet combing and hair detangling as well as good dry hair combing and improved manageability and styling.

By way of example, a combination of Necon DLD and behenyl amidopropyl dimethyl ethyl ammonium ethosulfate was made into a clear gel in water using Steareth-75 IPDI as the gelling and solubilizing agent. This mixture was found to be an excellent hair conditioner as well as providing thickening and gelling in hair conditioner and shampoo systems. In both of these products, the combination of Necon DLD, behenyl amidopropyl dimethyl ethyl ammonium ethosulfate and Steareth-75 IPDI provides greatly improved detangling and wet hair combing.

The following examples and studies provide illustrations of the product concepts. Note that Nequat and Necon products are available from Alzo International, Inc. Sayreville, N.J. USA.

| | Example 1 | Example 2 |
|---|---|---|
| Ingredients | % w/w | % w/w |
| Necon DLD (Dimethyllauramine Dimer | 20.0 | 20.0 |
| Dilinoleate) | | |
| Behenyl amidopropyl dimethyl ethyl | 15.0 | — |
| ammonium ethosulfate | | |
| Stearyl amidopropyl dimethyl ethyl | — | 15.0 |
| ammonium ethosulfate | | |
| Stearyl Alcohol | 65.0 | 65.0 |
| | 100.0 | 100.0 |

Examples 1 and 2 are solid, cream-white waxy materials that can be readily flaked or pastillated. Example 1 produces a hair conditioner that improves wet combing, detangling and reduces static flyaway. Example 2 makes hair conditioners that also improve wet combing, hair detangling while leaving a soft, smooth feeling on the hair.

| | Example 3 | Example 4 |
|---|---|---|
| Ingredients | % w/w | % w/w |
| Necon DLD (Dimethyllauramine Dimer | 20.0 | 20.0 |
| Dilinoleate) | | |
| Behenyl amidopropyl dimethyl ethyl | 7.0 | 15.0 |
| ammonium ethosulfate | | |
| Stearyl amidopropyl dimethyl ethyl | 8.0 | — |
| ammonium ethosulfate | | |
| Steareth-75 IPDI | — | 5.0 |
| Stearyl Alcohol | 65.0 | — |
| Deionized Water | — | 60.0 |
| | 100.0 | 100.0 |

Example 3 is a solid, cream-white waxy material that can be readily flaked or pastillated. Example 3 produces a hair conditioner that improves wet combing, detangling and reduces static flyaway, but leaves the hair feeling softer than Example 1. Example 4 is a clear stiff, gel that can be used as a hair conditioner as it is or it can used as a component in other hair conditioning formulas. It provides improved wet combing and hair detangling and leaves the hair feeling soft and conditioned when dry.
Example 1 was formulated into the following hair conditioner formulas (Example 5 and 7) and compared to a standard hair conditioner formula containing Behenyltrimethyl ammonium methosulfate (BTMS) (Example 6 and 8) and a similar formula containing Behenyltrimethyl ammonium chloride (BTC) (Example 9) in the following tests.

| | Example 5 | Example 6 |
|---|---|---|
| Ingredients | % w/w | % w/w |
| Example 1 | 4.00 | — |
| BTMS | — | 5.60 |
| Stearyl Alcohol | 2.00 | 2.00 |
| Microcare MTI (preservative) | 0.20 | 0.20 |
| Deionized Water | 93.80 | 92.20 |
| | 100.00 | 100.00 |

The tests were conducted as difference tests in which the combing and tactile properties of wet and dry tresses treated with the either Example 5, 6 or 7 above were compared. The columns presented in the tables below show the responses to each question by each panelist. The tests were conducted blind and all respondents were presented with two tresses to evaluate for each property and for each evaluation. They were asked to determine which of the two tresses they preferred for each property. The column marked % C.L. lists the confidence limit expressed as a % that was determined, based upon the number of responses, from Table 12 (pg 125) (Confidence limits for two tail difference test) found in “Sensory Testing” Mielgard, Vance and Civille, 1998.

The first test compares Example 5 to Example 6 [Nequat DBS compared to Behenyltrimethyl ammonium methosulfate (BTMS)] with pH as is (for Example 5 the pH is 6.8 and for Example 6 the pH is 4.3). The active quaternary basis for the two Examples is 1.4%.

TABLE 1
NEQUAT DBS V.S. BTMS
Example 5 v.s. Example 6

| | Example | | | | | | |
|---|---|---|---|---|---|---|---|
| | PS3-016A | | | PS3-016B | | | |
| | Nequat DBS | | | BTMS | | | |
| Samples | H | P | W | Q | E | X | % C.L. |
| WET COMB | | | | | | | |
| P S. | 1 | 1 | 1 | 0 | 0 | 0 | |
| A G. | 1 | 1 | 1 | 0 | 0 | 0 | |
| K S. | 1 | 0 | 1 | 0 | 1 | 0 | |
| J C. | 0 | 1 | 0 | 1 | 0 | 1 | |
| J M. | 1 | 0 | 1 | 0 | 1 | 0 | |
| E C. | 1 | 0 | 0 | 0 | 1 | 1 | |
| D B. | 1 | 1 | 0 | 0 | 0 | 1 | |
| J J. | 0 | 1 | 0 | 1 | 0 | 1 | |
| R P. | 1 | 1 | 1 | 0 | 0 | 0 | |
| L K. | 0 | 1 | 1 | 1 | 0 | 0 | |
| M D. | 0 | 1 | 1 | 1 | 0 | 0 | |
| T L. | 1 | 1 | 0 | 0 | 0 | 1 | |
| TOTAL SCORE | 8 | 9 | 7 | 4 | 3 | 5 | |
| AVERAGE SCORE | | 8 | | | 4 | | 90 |
| WET DETANGLING | | | | | | | |
| P S. | 0 | 1 | 1 | 1 | 0 | 0 | |
| A G. | 1 | 1 | 1 | 0 | 0 | 0 | |
| K S. | 1 | 1 | 0 | 0 | 0 | 1 | |
| J C. | 0 | 1 | 0 | 1 | 0 | 1 | |
| J M. | 1 | 0 | 1 | 0 | 1 | 0 | |
| E C. | 1 | 0 | 1 | 0 | 1 | 0 | |
| D B. | 1 | 0 | 0 | 0 | 1 | 1 | |
| J J. | 0 | 1 | 0 | 1 | 0 | 1 | |
| R P. | 1 | 1 | 1 | 0 | 0 | 0 | |
| L K. | 0 | 1 | 1 | 1 | 0 | 0 | |
| M D. | 0 | 1 | 1 | 1 | 0 | 0 | |
| T L. | 1 | 1 | 0 | 0 | 0 | 1 | |
| TOTAL SCORE | 7 | 9 | 7 | 5 | 3 | 5 | |
| AVERAGE SCORE | | 7.66667 | | | 4.33333 | | 80 |
| Wet Combing Score | 15 | 18 | 14 | 9 | 6 | 10 | |
| Wet Comb Percents | 62.5 | 75 | 58.33 | 37.5 | 25 | 41.67 | |
| Ave. Wet Comb | | 65.3 | | | 34.7 | | 95 |
| Percents | | | | | | | |
| Ave Wet Combing | | 7.83333 | | | 4.08333 | | |
| Score | | | | | | | |
| DRY COMB | | | | | | | |
| P S. | 1 | 0 | 1 | 0 | 1 | 0 | |
| A G. | 1 | 0 | 1 | 0 | 1 | 0 | |
| K S. | 0 | 1 | 1 | 1 | 0 | 0 | |
| J C. | 0 | 0 | 1 | 1 | 1 | 0 | |
| J M. | 0 | 0 | 0 | 1 | 1 | 1 | |
| E C. | 1 | 0 | 0 | 0 | 1 | 1 | |
| D B. | 0 | 1 | 1 | 1 | 0 | 0 | |
| J J. | 0 | 0 | 0 | 1 | 1 | 1 | |
| R P. | 1 | 1 | 1 | 0 | 0 | 0 | |
| L K. | 1 | 1 | 1 | 0 | 0 | 0 | |
| M D. | 0 | 1 | 1 | 1 | 0 | 0 | |
| T L. | 1 | 1 | 1 | 0 | 0 | 0 | |
| TOTAL SCORE | 6 | 6 | 9 | 6 | 6 | 3 | |
| AVERAGE SCORE | | 7 | | | 5 | | |
| FLY AWAY | | | | | | | |
| P S. | 1 | 1 | 1 | 0 | 0 | 0 | |
| A G. | 1 | 1 | 1 | 0 | 0 | 0 | |
| K S. | 1 | 1 | 1 | 0 | 0 | 0 | |
| J C. | 0 | 1 | 1 | 1 | 0 | 0 | |
| J M. | 0 | 0 | 0 | 1 | 1 | 1 | |
| E C. | 1 | 1 | 1 | 0 | 0 | 0 | |
| D B. | 1 | 0 | 0 | 0 | 1 | 1 | |
| J J. | 0 | 1 | 0 | 1 | 0 | 1 | |
| R P. | 1 | 1 | 1 | 0 | 0 | 0 | |
| L K. | 1 | 1 | 1 | 0 | 0 | 0 | |
| M D. | 0 | 1 | 1 | 1 | 0 | 0 | |
| T L. | 1 | 0 | 1 | 0 | 1 | 0 | |
| TOTAL SCORE | 8 | 9 | 9 | 4 | 3 | 3 | |
| AVERAGE SCORE | | 8.66667 | | | 3.33333 | | 95 |
| HAIR FEEL | | | | | | | |
| P S. | 0 | 0 | 1 | 1 | 1 | 0 | |
| A G. | 0 | 1 | 1 | 1 | 0 | 0 | |
| K S. | 1 | 1 | 1 | 0 | 0 | 0 | |
| J C. | 0 | 0 | 1 | 1 | 1 | 0 | |
| J M. | 1 | 1 | 1 | 0 | 0 | 0 | |
| E C. | 1 | 1 | 0 | 0 | 0 | 1 | |
| D B. | 0 | 0 | 0 | 1 | 1 | 1 | |
| J J. | 0 | 1 | 0 | 1 | 0 | 1 | |
| R P. | 1 | 1 | 1 | 0 | 0 | 0 | |
| L K. | 1 | 0 | 1 | 0 | 1 | 0 | |
| M D. | 0 | 1 | 1 | 1 | 0 | 0 | |
| T L. | 1 | 0 | 1 | 0 | 1 | 0 | |
| TOTAL SCORE | 6 | 7 | 9 | 6 | 5 | 3 | |
| AVERAGE SCORE | | 7.33333 | | | 4.66667 | | 70 |
| Dry Hair Score | 20 | 22 | 27 | 16 | 14 | 9 | |
| Dry Hair Percent | 55.56 | 61.12 | 75 | 44.44 | 38.88 | 25 | |
| Ave. Dry Hair Percentage | | 63.89 | | | 36.11 | | |
| Average Dry Hair | | 7.66667 | | | 4.33333 | | 99 |
| Score | | | | | | | |
| OVERALL SCORE | 35 | 40 | 41 | 25 | 20 | 19 | |
| AVG. OVERALL SCORE | 38.67 | | | 21.33 | | | |
| Percentages | | 64.44 | | | 35.56 | | |

### Procedure

1. Wash the hair tress with 5 g of PS2-077 Shampoo (massage for 1 min. and then rinse for 1 min.);
2. Blot dry with paper towels;
3. Place into weigh boat. Weight out 2.5 g of conditioners. Massage into the tress for 1 min.);
4. Wash in warm water for 1 min;
5. Blot dry with paper towels.
6. Perform Wet evaluation;
7. Blow dry the hair tresses;
8. Perform Dry evaluations.
   This test shows that the Example 7 conditioner [(made with the Example 1 containing Dimethyllauramine Dimer Dilinoleate and Behenyl amidopropyl dimethyl ethyl ammonium ethosulfate (Nequat DBS)] is preferred for wet hair detangling and wet hair combing. In addition, the flyaway was found to be significantly better for the Example 5 conditioner.
   The next tests compare Examples 7, 8, 9 and 10 with the pH adjusted to similar values (approximately 4.3 to 4.5) using citric acid and with the active ingredient levels adjusted to about 1.0%.

| | Example 7 | Example 8 | Example 9 | Example 10 |
|---|---|---|---|---|
| Ingredients | % w/w | % w/w | % w/w | % w/w |
| Example 1 | 2.86 | — | — | — |
| BTMS (~25% actives) | — | 4.00 | — | — |
| BTC (~25% actives) | — | — | 4.00 | — |
| Distearyl Dimonium | — | — | — | 1.08 |
| Chloride | | | | |
| Stearyl Alcohol | 1.14 | — | — | 2.92 |
| Microcare MTI | 0.20 | 0.20 | 0.20 | 0.20 |
| (preservative) | | | | |
| Deionized Water | 95.80 | 95.80 | 95.80 | 95.80 |
| | 100.00 | 100.00 | 100.00 | 100.00 |

This test again shows that Nequat DBS (as used in Example 7) is preferred for wet combing and detangling over a similar formula made with Behentrimonium Methosulfate.
The following test compares Examples 7 and 9. Both Examples were again adjusted to similar pH values (approximately 4.3 to 4.5) using citric acid and the Examples have similar active ingredient levels of about 1.0%.

NEQUAT DBS v.s. BTMS

| FOR | | | | | | | | | |
|---|---|---|---|---|---|---|---|---|---|
| Blend | DBS | TMS | TMS | DBS | TMS | DBS | TMS | DBS | |
| Samples | K | W | U | C | F | R | H | T | C.L. |
| WET COMB | | | | | | | | | |
| P S. | 1 | 0 | 0 | 1 | 1 | 0 | 0 | 1 | |
| K S. | 1 | 0 | 0 | 1 | 1 | 0 | 1 | 0 | |
| R K | 0 | 1 | 0 | 1 | 1 | 0 | 1 | 0 | |
| J C | 1 | 0 | 0 | 1 | 0 | 1 | 0 | 1 | |
| J M. | 1 | 0 | 0 | 1 | 1 | 0 | 0 | 1 | |
| L C. | 1 | 0 | 0 | 1 | 0 | 1 | 0 | 1 | |
| D B. | 1 | 0 | 0 | 1 | 1 | 0 | 0 | 1 | |
| J J. | 0 | 1 | 0 | 1 | 1 | 0 | 0 | 1 | |
| R P. | 1 | 0 | 0 | 1 | 0 | 1 | 0 | 1 | |
| L K. | 1 | 0 | 0 | 1 | 1 | 0 | 1 | 0 | |
| C Z. | 0 | 1 | 1 | 0 | 1 | 0 | 0 | 1 | |
| T. | 0 | 1 | 0 | 1 | 1 | 0 | 0 | 1 | |
| TOTAL SCORE | 8 | 4 | 1 | 11 | 9 | 3 | 3 | 9 | |
| AVERAGE SCORE | NEQUAT DBS | 7.75 | | | B-TMS | 4.25 | | | 90% |
| WET DETANGLING | | | | | | | | | |
| P S | 1 | 0 | 0 | 1 | 1 | 0 | 0 | 1 | |
| K S. | 1 | 0 | 0 | 1 | 0 | 1 | 1 | 0 | |
| R K. | 0 | 1 | 0 | 1 | 1 | 0 | 1 | 0 | |
| J C | 1 | 0 | 0 | 1 | 0 | 1 | 0 | 1 | |
| J M. | 1 | 0 | 0 | 1 | 1 | 0 | 1 | 0 | |
| L C. | 1 | 0 | 0 | 1 | 0 | 1 | 0 | 1 | |
| D B. | 1 | 0 | 0 | 1 | 1 | 0 | 0 | 1 | |
| J J. | 0 | 1 | 0 | 1 | 1 | 0 | 0 | 1 | |
| R P. | 1 | 0 | 0 | 1 | 0 | 1 | 0 | 1 | |
| L K. | 1 | 0 | 0 | 1 | 1 | 0 | 0 | 1 | |
| C Z. | 0 | 1 | 1 | 0 | 1 | 0 | 0 | 1 | |
| T L. | 0 | 1 | 0 | 1 | 1 | 0 | 1 | 0 | |
| TOTAL SCORE | 8 | 4 | 1 | 11 | 8 | 4 | 4 | 8 | |
| AVERAGE SCORE | NEQUAT DBS | 7.75 | | | B-TMS | 4.25 | | | 90% |
| DRY COMB | | | | | | | | | |
| P S. | 1 | 0 | 0 | 1 | 1 | 0 | 0 | 1 | |
| K S. | 1 | 0 | 0 | 1 | 1 | 0 | 1 | 0 | |
| R K. | 0 | 1 | 0 | 1 | 1 | 0 | 0 | 1 | |
| J C. | 0 | 1 | 1 | 0 | 1 | 0 | 1 | 0 | |
| J M. | 0 | 1 | 0 | 1 | 1 | 0 | 0 | 1 | |
| L C. | 0 | 1 | 0 | 1 | 0 | 1 | 1 | 0 | |
| D B. | 1 | 0 | 0 | 1 | 0 | 1 | 1 | 0 | |
| J J. | 0 | 1 | 1 | 0 | 1 | 0 | 1 | 0 | |
| R P. | 0 | 1 | 0 | 1 | 0 | 1 | 0 | 1 | |
| L K. | 1 | 0 | 0 | 1 | 1 | 0 | 0 | 1 | |
| C Z. | 1 | 0 | 1 | 0 | 1 | 0 | 0 | 1 | |
| T L. | 1 | 0 | 0 | 1 | 1 | 0 | 1 | 0 | |
| TOTAL SCORE | 6 | 6 | 3 | 9 | 9 | 3 | 6 | 6 | |
| AVERAGE SCORE | NEQUAT DBS | 6 | | | B-TMS | 6 | | | |
| FLY AWAY | | | | | | | | | |
| P S. | 1 | 0 | 0 | 1 | 1 | 0 | 0 | 1 | |
| K S. | 1 | 0 | 1 | 0 | 1 | 0 | 1 | 0 | |
| R K. | 0 | 1 | 0 | 1 | 1 | 0 | 0 | 1 | |
| J C. | 1 | 0 | 0 | 1 | 1 | 0 | 0 | 1 | |
| J M. | 0 | 1 | 0 | 1 | 1 | 0 | 0 | 1 | |
| L C. | 0 | 1 | 0 | 1 | 0 | 1 | 1 | 0 | |
| D B. | 1 | 0 | 0 | 1 | 0 | 1 | 0 | 1 | |
| J J. | 1 | 0 | 0 | 1 | 1 | 0 | 1 | 0 | |
| R P. | 0 | 1 | 0 | 1 | 0 | 1 | 0 | 1 | |
| L K. | 0 | 1 | 0 | 1 | 0 | 1 | 0 | 1 | |
| C Z. | 1 | 0 | 1 | 0 | 1 | 0 | 0 | 1 | |
| T L. | 1 | 0 | 0 | 1 | 1 | 0 | 1 | 0 | |
| TOTAL SCORE | 7 | 5 | 2 | 10 | 8 | 4 | 4 | 8 | |
| AVERAGE SCORE | NEQUAT DBS | 7.25 | | | B-TMS | 4.75 | | | |
| HAIR FEEL | | | | | | | | | |
| P S. | 1 | 0 | 0 | 1 | 1 | 0 | 0 | 1 | |
| K S. | 0 | 1 | 0 | 1 | 0 | 1 | 1 | 0 | |
| R K. | 0 | 1 | 1 | 0 | 1 | 0 | 1 | 0 | |
| J C. | 1 | 0 | 0 | 1 | 1 | 0 | 1 | 0 | |
| J M. | 0 | 1 | 0 | 1 | 1 | 0 | 0 | 1 | |
| L C. | 0 | 1 | 0 | 1 | 0 | 1 | 1 | 0 | |
| D B | 0 | 1 | 1 | 0 | 0 | 1 | 0 | 1 | |
| J J. | 1 | 0 | 0 | 1 | 0 | 1 | 1 | 0 | |
| R P. | 0 | 1 | 0 | 1 | 0 | 1 | 0 | 1 | |
| L K. | 1 | 0 | 0 | 1 | 0 | 1 | 0 | 1 | |
| C Z. | 1 | 0 | 1 | 0 | 1 | 0 | 0 | 1 | |
| T L. | 0 | 1 | 0 | 1 | 0 | 1 | 1 | 0 | |
| TOTAL SCORE | 5 | 7 | 3 | 9 | 5 | 7 | 6 | 6 | |
| AVERAGE SCORE | NEQUAT DBS | 6.25 | | | B-TMS | 5.25 | | | |

This test again shows that Nequat DBS (as used in Example 7) is significantly preferred for wet combing and detangling over a similar formula made with Behenyltrimethyl ammonium chloride.
Example 4 was evaluated by comparing it to Example 6 for wet combing and wet hair detangling using an informal comparison of several treated tresses and it was found to be better in both categories. This product makes an excellent hair as is or when diluted by two or three to one. Additionally, this product can be used in shampoos as illustrated by the following Example 11.

### Example 11

Nequat DBS-WS Baby Shampoo ps3-049

| PRODUCT NAME | INCI | W/W % | Supplier | 500.00 |
|---|---|---|---|---|
| Ro water | water | 61.70 | | 308.50 |
| Edeta BD | Disodium EDTA | 0.10 | BASF | 0.50 |
| Mackam CB-35 | Coco-Betaine | 6.00 | McIntyre | 30.00 |
| Mackamine CAO | Cocamidopropylamine Oxide | 5.00 | McIntyre | 25.00 |
| Amphosol CS-50 | Cocamidopropyl Hydroxysultaine | 6.00 | Stepan | 30.00 |
| Plantaren 1200 | Alkyl Glucosides | 5.00 | Cognis | 25.00 |
| Plantaren 1300 | Lauryl Glucoside | 10.00 | Cognis | 50.00 |
| Dermothix 75 | Disteareth-75 IPDI | 1.00 | ALZO | 5.00 |
| Polyderm PPI-SI-WI | Bis-PPG-12 Dimethicone IPDI | 2.00 | ALZO | 10.00 |
| Nequat DBS-WS | Water (&) Behenamidopropyl | 2.00 | ALZO | 10.00 |
| | Ethyldimonium Ethosulfate (&) | | | |
| | Dimethyl Lauramine Dimer | | | |
| | Dilinoleate (&) Disteareth-75 IPDI | | | |
| Polyderm PPI-SI-WS | Bis-PEG-12 Dimethicone IPDI | 0.50 | ALZO | 2.50 |
| Fragrance | CARRUBBA Melon | 0.50 | CARRUBBA | 2.50 |
| Microcare MTI | Methylisothiazolinone (&) | 0.20 | ACTIchem | 1.00 |
| | Iodopropynyl butylcarbamate | | | |
| | TOTAL | 100.00 | | |

pH =
Viscosity: LV3@6 rpm

### Manufacturing Procedure:

1. Into a vessel suitable for making shampoos add water+Edeta BD and begin heating the batch to T=75° C. w/agitation.
2. When the batch becomes clear add the following ingredients in order w/ample mixing* between additions: Mackam CB-35=>Amphosol CS-50=>Mackamine CO=>Plantaren 2000=>Plantaren 1300=>Dermothix-75. When the batch reaches 60° C. add Polyderm PPI-SI-WI and mix until dissolved. * ample mixing—mixing until the ingredient dissolves or batch becomes homogenous.
3. At 60° C. add Nequat DBS-WS and mix until dissolved.
4. Begin cooling the batch. At 50° C. add Pre-Mix of PPI-Si-WS+Fragrance.
5. At 45° C. add pre-mix of Microcare MTI mix until homogenous.
*The foregoing information, accurate to the best of our knowledge, is intended to be helpful but no warranty is expressed or implied as to the results obtained from use of the information, procedure or products suggested herein. Neither is any permission or recommendation to practice any invention covered by patent either expressed or implied.

### Example 12

### Cationic Cream (Emulsion)

| Product Name | INCI | W/W % | Supplier |
|---|---|---|---|
| RO Water | Aqua | 78.20 | |
| Glycerine | Glycerol | 7.65 | ALZO |
| Part A | | | |
| Nequat DBS | Stearyl Alcohol (&) | 4.75 | ALZO |
| | Stearamidopropyl | | |
| | Ethyldimonium | | |
| | Ethosulfate (&) | | |
| | Dimethyl Lauramine (&) | | |
| | Dimer Acid | | |
| Pinnacle 225 | Petrolatum | 4.55 | Avatar |
| Dermol IPP | Isopropyl Palmitate | 4.25 | ALZO |
| DC 200 Fluid | Dimethicone | 0.40 | Dow Corning |
| (1000 cst) | | | |
| Microcare MTI | Methylisothiazolinone (&) | 0.20 | ACTI-chem |
| | Iodopropynyl | | |
| | Butylcarbamate | | |
| | Total | 100.00 | |

### Manufacturing Procedure

1. Into a vessel suitable for making creams and lotions add water+Glycerine and begin heating batch to T=80 C w/agitation.
2. In a separate vessel combine Part A ingredients. Heat to 80 C w/Mixing.
3. When Part A becomes clear and reaches 80 C add it to the main batch and mix for 20 min. maintaining the temperature.
4. Add Dimethicone at 70 C and homogenize for 4 min @ 3000 rpm. Begin cooling the batch.
5. At 45 C add Microcare and mix until homogenous.

What we claim is:

1. A composition comprising a dicarboxylic acid complexed with a tertiary monofattytrialkylamine (dimer acid amine complex or salt) in a molar ratio of about 1:2, wherein said monofattytrialkylamine has the formula I: 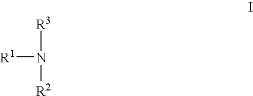
Where R¹ is a C₈ to C₂₄ alkyl group (preferably a C₁₂ to C₁₈ alkyl group); and
R² and R³ are each independently a C₁-C₃ alkyl group, to form a compound according to formula II: 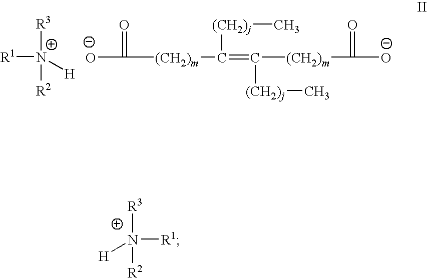
Where R′, R² and R³ are the same as above; and each m is from 4 to 12, preferably 6-10, most preferably 7, and each j is from 4 to 12, most preferably 8; and
A quaternary ammonium conditioner having at least one C₈-C₂₀ fatty alkyl group;
Wherein the weight ratio of said dicarboxylic acid amine salt compound of formula II to said quaternary amine conditioner in said composition ranges from about 10:1 to about 1:10, and said composition optionally comprises an effective amount of cosmetically acceptable thickener and/or water.

2. The composition according to claim 1 wherein R¹ is a C₁₂ alkyl group, R² and R³ are each methyl groups; m is 7 and j is 8.

3. The composition according to claim 1 or 2 wherein the weight ratio of said dicarboxylic acid amine salt compound and said quaternary amine conditioner in said composition ranges from about 3:1 to about 1:3.

4. The composition according to either of claim 1 or 3 wherein said dicarboxylic acid is dimer acid.

5. The composition according to any of claims 1-4 further including water and an effective amount of a C₁₄-C₂₂ alkyl end-capped polyethyleneoxide polyurethane thickener.

6. The composition according to claim 5 wherein said thickener is disteareth 75 isophorone diurethane or disteareth100 isophorone diurethane.

7. The composition according to any of claims 1-6 wherein said dicarboxylic acid is dimer dilinoleic acid.

8. The composition according to any of claims 1-7 wherein said quaternary amine conditioner has the structure: 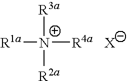
Where R^{1a} is a —(CH₂)ₙ—N_{y}—Z group;
n is 0, 1, 2, 3, 4, 5, 6, 7 or 8;
y is 0 or 1, with the proviso that y is 0 when n is 0; and
Z is a C₈-C₂₄ acyl group or alkyl group, preferably an acyl group;
R^{2a} and R^{3a} and each independently a C₁-C₃ alkyl group;
R^{4a} is a group formed by reacting R^{1a}R^{2a}R^{3a}N with a quaternizing agent selected from the group consisting of dimethyl sulfate, diethyl sulfate, methyl bromide, benzyl chloride, ethyl benzyl chloride, methyl benzyl chloride, dichloroethyl ether, epichlorohydrin, ethylene chlorohydrin, methyl chloride and allyl chloride
to form an N—R^{4a} group with the amine and the resulting positively charged quaternary amine group is complexed with an anionic group or counterion, which is represented as X⁻.

9. The composition according to claim 8 wherein X⁻ is an anionic chloride, bromide, iodide, fluoride, carboxylate, mono- or di-anion sulfate or mono-, di- or tri-anion phosphate.

10. The composition according to claim 8 or 9 wherein X⁻ is an anionic chloride or ethosulfate.

11. The composition according to any of claims 1-10 wherein said quarternary amine surfactant is isostearyl dimethylethylammonium ethosulfate, behenyl aminopropyldimethylethylammonium ethosulfate or mixtures thereof.

12. The composition according to any of claims 1-4 and 6-11 in the form of a dry powder.

13. The composition according to any of claims 1-4 and 6-11 in combination with water, ethanol, isopropanol or mixtures thereof.

14. The composition according to any of claims 1-13 wherein said ratio of said dicarboxylic acid to said tertiary monofattytrialkylamine in said dicarboxylic acid amine salt is 1:2.

15. A personal care composition comprising a composition according to any of claims 1-14 in combination with at least one additional component selected from the group consisting of fragrances, emollients, solvents/diluents, opacifiers, sunscreen agents, anti-perspirants, deodorizers, antiperspirants, antimicrobial agents, dyes, pigments, foaming agents, gelling agents, solubilizing agents, humectants, stiffening agents and mixtures thereof.

16. The personal care composition according to claim 15 which is a floating bath oil, an after shave, a cream, a lotion, a deodorant, a pre-electric shave lotion, an after-shave lotion, an antiperspirant, a shampoo, conditioner, rinse, skin care products, eye makeups, body shampoos, protective skin formulations, lipsticks, lip glosses, after-bath splashes, presun and sun products.

17. The composition according to claim 15 wherein said personal care composition is a shampoo, conditioner or rinse.

18. An emulsion comprising water, an oil and an effective amount of a composition according to any of claims 1-14.

19. A method of improving wet hair tangling and wet hair combining in a shampoo, condition or rinse comprising adding to said shampoo, conditioner or rinse an effective amount of a composition according to any of claims 1-14.
